SEQUENCE LISTING (43 sequences)

SEQ ID NO: 1 - protein, 8 aa, synthetic construct
GPLGIAGQ

SEQ ID NO: 2 - protein, 6 aa, synthetic construct
PVGLIG

SEQ ID NO: 3 - protein, 6 aa, synthetic construct
PLGLAG

SEQ ID NO: 4 - protein, 14 aa, synthetic construct
GGGVPLSLYSGGGG

SEQ ID NO: 5 - protein, 10 aa, synthetic construct
VLVPMAMMAS

SEQ ID NO: 6 - protein, 10 aa, synthetic construct
GPLGMRGLGK

SEQ ID NO: 7 - protein, 10 aa, synthetic construct
VPLSLYSGCG

SEQ ID NO: 8 - protein, 8 aa, synthetic construct
GQPGIFGQ

SEQ ID NO: 9 - protein, 8 aa, synthetic construct
RPLALWRS

SEQ ID NO: 10 - protein, 8 aa, synthetic construct
GPVGLIGK

SEQ ID NO: 11 - protein, 8 aa, synthetic construct
HPVGLLAR

SEQ ID NO: 12 - protein, 7 aa, synthetic construct
DGPLGVC

SEQ ID NO: 13 - protein, 7 aa, synthetic construct
GPQGIAG

SEQ ID NO: 14 - protein, 7 aa, synthetic construct
GRIGFLR

SEQ ID NO: 15 - protein, 6 aa, synthetic construct
RIGFLR

SEQ ID NO: 16 - protein, 5 aa, synthetic construct
GPLGV

SEQ ID NO: 17 - protein, 5 aa, synthetic construct
PLGVR

SEQ ID NO: 18 - protein, 9 aa, synthetic construct
GPQGIAGQR

SEQ ID NO: 19 - protein, 4 aa, synthetic construct
PLGL

SEQ ID NO: 20 - protein, 6 aa, synthetic construct
PLGLWA

SEQ ID NO: 21 - protein, 7 aa, synthetic construct
GPLGVRG

SEQ ID NO: 22 - protein, 7 aa, synthetic construct
PLGLAAV

SEQ ID NO: 23 - protein, 6 aa, synthetic construct
GPILGR

SEQ ID NO: 24 - protein, 7 aa, synthetic construct
PRVRLLG

SEQ ID NO: 25 - protein, 7 aa, synthetic construct
APLANLG

SEQ ID NO: 26 - protein, 7 aa, synthetic construct
PLAVNLG

SEQ ID NO: 27 - protein, 9 aa, synthetic construct
ILGRLVREG

SEQ ID NO: 28 - protein, 8 aa, synthetic construct
PLARSVLG

SEQ ID NO: 29 - protein, 8 aa, synthetic construct
PILGIVGR

SEQ ID NO: 30 - protein, 10 aa, synthetic construct
LRVSLGIVGR

SEQ ID NO: 31 - protein, 7 aa, synthetic construct
PGVRVIG

SEQ ID NO: 32 - protein, 8 aa, synthetic construct
PGILGISR

SEQ ID NO: 33 - protein, 6 aa, synthetic construct
PLGILL

SEQ ID NO: 34 - protein, 8 aa, synthetic construct
PQILGISR

SEQ ID NO: 35 - protein, 5 aa, synthetic construct
PGILL

SEQ ID NO: 36 - protein, 6 aa, synthetic construct
PLRLVS

SEQ ID NO: 37 - protein, 7 aa, synthetic construct
PRVRLLG

SEQ ID NO: 38 - protein, 8 aa, synthetic construct
CREIIGHR

SEQ ID NO: 39 - protein, 5 aa, synthetic construct
RRRRR

SEQ ID NO: 40 - protein, 7 aa, synthetic construct
KLEGGVR

SEQ ID NO: 41 - protein, 5 aa, synthetic construct
WHHLR

SEQ ID NO: 42 - protein, 6 aa, synthetic construct
RRRFMW

SEQ ID NO: 43 - protein, 12 aa, synthetic construct
RHRHRHRHRHRK

(19) United States
(12) Patent Application Publication
(10) Pub. No.: US 2026/0273094 A1
(43) Pub. Date: Sep. 17, 2026

(54) QUANTITATIVE MONITORING OF DRUG RELEASE BY ENZYME-RESPONSIVE POLYMERIC NANOPARTICLE

(71) Applicant: Board of Trustees of Michigan State University, East Lansing, MI (US)

(72) Inventors: Bryan SMITH, East Lansing, MI (US); Romila MANCHANDA, East Lansing, MI (US)

(73) Assignee: Board of Trustees of Michigan State University, East Lansing, MI (US)

(21) Appl. No.: 19/166,257

(22) PCT Filed: May 6, 2024
(86) PCT No.: PCT/US2024/028073

§ 371 Date: Sep. 17, 2025

Related U.S. Application Data
(60) Provisional application No. 63/464,209, filed on May 5, 2023.

(51) Int. Cl. A61K 47/69; A61K 31/541; A61K 31/65; A61K 31/704

(52) U.S. Cl. CPC A61K 47/6937; A61K 31/541; A61K 31/65; A61K 31/704

(57) ABSTRACT

Disclosed herein is a nanocomposite particle comprising a polymer, a cleavable entity conjugated to the polymer, a magnetic nanoparticle, and a therapeutically effective amount of an active agent, and pharmaceutical compositions comprising the nanocomposite particle. MPI with the disclosed nanocomposite particles also allows for real-time quantitative monitoring of the rate of release of the active agent loaded onto the nanocomposite particle anywhere in the body and at any depth. The nanocomposite particle is also useful for the treatment of cancer.

### CROSS-REFERENCE TO RELATED APPLICATIONS

This application claims the benefit of U.S. Provisional Application No. 63/464,209 filed on May 5, 2023. The entire disclosure of the application identified in this paragraph is incorporated herein by reference.

### STATEMENT OF A SEQUENCE LISTING

The present disclosure contains references to amino acid sequences and nucleic acid sequences which have been submitted concurrently herewith as the sequence listing .xml file entitled “000462wopoa_SequenceListing.xml,” file size 38,068 bytes, created on 4 May 2024. The aforementioned sequence listing is hereby incorporated by reference in its entirety pursuant to 37 C.F.R. § 1.52(e)(5).

### FIELD

The field of the invention relates to a nanocomposite particle comprising a cleavable entity-polymer conjugate, magnetic nanoparticles, and an active agent.

### BACKGROUND

The background description includes information that may be useful in understanding the compositions and methods described herein. It is not an admission that any of the information provided herein is prior art or relevant to the compositions and methods, or that any publication specifically or implicitly referenced is prior art.

In clinical oncology, surgery and chemotherapy are primary treatment methods. However, cancer metastasis is a major problem behind treatment failure and death. In this context, malignancy and metastatic potential have been correlated with matrix metalloprotease (MMP) production in tumor as MMPs are overexpressed in most cancers. For example, MMP-2 affects several cellular pathways to increase cell proliferation, motility, invasion, and angiogenesis, ultimately resulting in alterations in the tumor that can drive metastatic activity. Thus, proteases comprise key therapeutic and diagnostic targets for the treatment, detection, and prognosis of cancers. MMPs have a wide range of natural substrates, mostly proteins or protein derivatives, including ECM proteins (e.g. collagen, gelatin, fibronectin, etc.) and other proteins (e.g. fibrinogen). Different MMPs target specific proteins. For instance, MMP-2 cleaves most collagen types, except for collagen-6 and -9. The cleavability of MMP substrates is significantly dependent on their amino acid sequences, especially the region around the site of cleavage.

Nanocomposite particles have distinct advantages in pharmaceutical applications due to their small, tunable size and the ability to adapt to specific design needs, including sensing coupled with therapy. Despite the numerous advantages of polymer nanocarriers, drug release from the polymer nanocarrier is uncontrolled at the desired site and current nano-approaches cannot provide quantitative feedback regarding the amount of drug delivered to the desired site. Improvements are needed to provide controlled release with quantitative feedback regarding the amount of drug delivered to the target side.

Molecular imaging facilitates visualization and quantification of processes happening at cellular and molecular level. Nanocomposite particles that can serve as both a drug delivery carrier and an imaging agent provide real-time quantification of the released drug. Current drug release monitoring technologies, such as optical and magnetic resonance imaging, are not linearly quantitative, show signal convolution decreases reliability, and/or display insufficient penetration depth. Magnetic particle imaging (MPI) is a non-invasive imaging method which utilizes superparamagnetic nanoparticles (e.g., iron oxide) as imaging agent wherein signal intensity can be quantified with high spatial and temporal resolution. Due to its properties such as large imaging depth, high sensitivity, and capacity for real time imaging, MPI has the potential to be used for quantitative monitoring the drug release.

### SUMMARY

This section provides a general summary of the disclosure and is not a comprehensive disclosure of its full scope or all of its features.

Leveraging a synergistic integration of advanced techniques such as MPI and a smart nano-approach, the nanocomposite particle described herein is useful, for example, for tumor therapy as dual enzyme-responsive drug delivery carrier and drug release monitoring imaging agent, as shown in FIG. 1. MPI with the disclosed nanocomposite particles allows for real-time monitoring of the location of the nanocomposite particle release of the active agent loaded onto the nanocomposite particle. MPI with the disclosed nanocomposite particles also allows for real-time quantitative monitoring of the rate of release of the active agent loaded onto the nanocomposite particle anywhere in the body and at any depth.

Disclosed herein is a nanocomposite particle comprising a cleavable entity-polymer conjugate, magnetic nanoparticle, and an active therapeutic agent and pharmaceutical compositions comprising the nanocomposite particle.

Also disclosed herein is a method for treating a disease, disorder, or condition in a subject comprising administration of a therapeutically effective amount of the nanocomposite particle. The disease, disorder, or condition may be cancer.

### BRIEF DESCRIPTION OF THE DRAWINGS

FIG. 1 depicts an exemplary preparation and delivery process of MMP-2 active (DNCa) and MMP-2 inactive (DNCi) nanocomposite particles.

FIG. 2A depicts the UV-Vis spectroscopy of a polymer (PLGA) in DMSO and a cleavable entity polymer conjugate (PLGA-SEQ ID NO: 1) in DMSO or water. FIG. 2B depicts a DLS image of DNCa and FNCa. FIG. 2C depicts the zeta-potential of DNCa (−10.87 mV) and FNCa (−22.43 mV).

FIG. 3A depicts time dependent doxorubicin (DOX) release from DNCa at varying enzyme conditions. FIG. 3B depicts an MPI signal change and DOX release from DNCa at 0.5 mg/mL MMP-2. FIG. 3C depicts the correlation curve of DOX and MPI signal intensity at 0.5 mg/mL MMP-2 (r²=0.977). The error bars in FIGS. 3A and 3B represent standard deviation.

FIG. 4A shows an SEM image of DNCa incubated in phosphate buffer (pH 6.5) with and without MMP-2 (0.5 mg/mL) for 8 hr. FIG. 4B shows an SEM image of DNCa incubated in phosphate buffer (pH 6.5) with and without MMP-2 (0.5 mg/mL) for 48 hr.

FIG. 5A depicts the percentage increase in MPI signal in a tumor site in mice over 96 hrs. FIG. 5B shows the body weight of the mice during imaging. FIG. 5C depicts the tumor volume during in mice treated with nanocomposite particles comprising a cleavable entity (DNCa) or an uncleavable entity (DNCi). The error bars represent standard deviation.

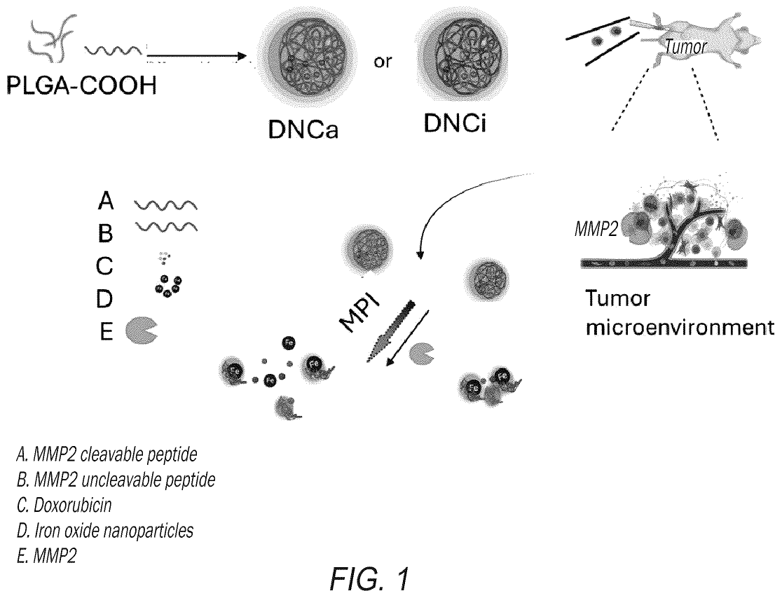

### DETAILED DESCRIPTION

### Definitions

As used herein, “nanocomposite particle” refers to a composition comprising a polymer, a cleavable entity conjugated to the polymer (cleavable entity-polymer conjugate), magnetic nanoparticles, and an active agent. The nanocomposite particle has an average diameter of about 50 nm to about 5,000 nm (5 μm). The nanocomposite particle may have a maximum average diameter of up to about 4,000 nm (4 μm), up to about 3,000 nm (3 μm), up to about 2,000 (2 μm), up to about 1,000 nm (1 μm), up to about 900 nm, up to about 800 nm, up to about 700 nm, up to about 600 nm, up to about 500 nm, up to about 400 nm, or up to about 300 nm. In some embodiments, the nanocomposite polymer may have an average diameter between about 50 nm and about 500 nm, between about 60 nm and about 400 nm, between about 70 nm to about 300 nm, between about 80 nm and about 200 nm, and/or between about 90 nm and about 120 nm. The size of the nanocomposite particle described herein may be varied by changing or altering the polymer length, mixing time, and/or the ratio of organic and aqueous phases. The nanocomposite particle may be administered to a subject as described to treat or prevent a disease, disorder, or condition.

“Subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In certain embodiments, the subject can be human (e.g., adult male, adult female, adolescent male, adolescent female, male child, female child) under the care of a physician or other health worker. In certain embodiments the subject may not be under the care of a physician or other health worker.

“Treat” and “treatment” each refer to a method for reducing, inhibiting, or otherwise ameliorating an infection by administering a therapeutic to a subject in need of treatment. In some embodiments, the subject in need of treatment may include a subject having, diagnosed as having, or suspected to have, an infection. In a particular embodiment, treat or treatment includes administering a therapeutic to a subject having, diagnosed as having, or suspected of having cancer. In some embodiments, the subject may be asymptomatic. Treatment includes administration of a nanocomposite particle described herein.

“Effective amount” or “therapeutically effective amount” refers to the amount and/or dosage, and/or dosage regime of one or more agent(s) necessary to bring about the desired result e.g., an amount sufficient to reduce the occurrence of cancer or cancer metastasis and/or an amount sufficient to treat cancer or cancer metastasis in a subject.

The term “cancer” includes, but is not exclusive of breast, brain, pancreas, prostate, ovary, lung, brain, bladder, testicle, lymph and lymph nodes, muscle, liver, skin, mouth, intestine, colon, and stomach. The term further encompasses cancer metastasis.

The term “injection” refers to a method of administration where the composition is administered to the body via needle. In an embodiment, the injection is selected from the group consisting of subcutaneous injection, intramuscular injection, intravenous injection, infusion, intraperitoneal injection, intrapleural injection, intrapericardial injection, intrathecal injection, intra-arterial injection, intravesical injection, and intralesional injection. The injection can be a single injection, including an acute injection or a continuous injection. The injection can be delivered over a number of days in one or multiple injections.

As used herein, the term “polymer” refers to compounds comprising multiple subunits (monomers). In some embodiments, the polymer may be a biodegradable polymer, including a polymer that breaks down (degrades) when located within a biological environments such as a body, organ, tissue, tumor, group of cells, extracellular matrix (ECM), an in vivo system, or an in vitro system that mimics a biological environment. The polymer may be conjugated to a peptide, e.g., a cleavable entity, and further combined with a magnetic nanoparticle and an active agent such that the components aggregate to form the nanocomposite particle. Degradation of the polymer leads to the disintegration of the nanocomposite particle and, subsequently, release of the magnetic nanoparticles and the active agent into the local environment.

The polymer of the disclosed nanocomposite particle is not particularly limited and may include, for example, poly(lactic-co-glycolic acid) (PLGA), polyethylenimine (PEI), PLGA-NH2, chitosan, polyglycolic acid (PGA), polylactic acid (PLA), poly(caprolactone), polyethylene glycol (PEG), polyhydroxybutyrate, hyaluronic acid, polymethylmethacrylate, polyethylmethacrylate, polypropylene, collagen, gelatin, albumin, alginate, poly(methacrylic acid), poly(acid acrylic), poly(anhydrides), PEG-PLA, PLGA-PEG-COOH, and/or poly(propylene sulfide) (PPS). Where the polymer includes two different monomer subunits (e.g., PLGA, which includes both lactic acid and glycolic acid monomers), the ratio of the two subunits may be about 95:5, about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, and about 5:95. In a particular embodiment, the polymer may be PLGA having a molecular weight of between about 1,000 Da and about 250,000 Da (e.g., between about 1 kDa and about 250 kDa), such as acid-terminated PLGA having a lactide:glycolide ratio of about 65:35 and a molecular weight of about 24-38 kDa, acid-terminated PLGA having a lactide:glycolide ratio of about 75:25 and a molecular weight of about 75-115 kDa, PLGA having a lactide:glycolide ratio of about 50:50 and a molecular weight of about 4.2 kDa, PLGA having a lactide:glycolide ratio of about 50:50 and a molecular weight of about 24-38 kDa, PLGA having a lactide:glycolide ratio of about 50:50 and a molecular weight of about 38-54 kDa, poly(D,L-lactide-co-glycolide)-b-poly(ethylene glycol)-carboxylic acid having a lactide:glycolide ratio of about 85:15 and a molecular weight of about 5-13 kDa, polyglycolic acid/80:10 or polylactic acid:polyglycolic acid/90:10.

As used herein, “cleavable entity” refers to an amino acid sequence which may be cleaved (e.g., cut) by an enzyme. The enzyme may be any enzyme having peptidase, protease, or proteinase activity. For example, the enzyme may be a matrix metalloprotease (MMP) or a disintgrin metalloproteinase with a thrombospondin motive (ADAMTS). In a particular embodiment, the MMP may be one of MMP-1, MMP-2, MMP-7, MMP-9, MMP-13, MMP-14, MMP-15, and MTI-MMP. Although the cleavable entity sequence is not particular limited, the cleavable entity may have a sequence having at least 75% sequence identity to GPLGIAGQ (SEQ ID NO: 1), PVGLIG (SEQ ID NO: 2), PLGLAG (SEQ ID NO: 3), GGGVPLSLYSGGGG (SEQ ID NO: 4), VLVPMAMMAS (SEQ ID NO: 5), GPLGMRGLGK (SEQ ID NO: 6), VPLSLYSGCG (SEQ ID NO: 7), GQPGIFGQ (SEQ ID NO: 8), RPLALWRS (SEQ ID NO: 9), GPVGLIGK (SEQ ID NO: 10), HPVGLLAR (SEQ ID NO: 11), DGPLGVC (SEQ ID NO: 12), GPQGIAG (SEQ ID NO: 13), GRIGFLR (SEQ ID NO: 14), RIGFLR (SEQ ID NO: 15), GPLGV (SEQ ID NO: 16), PLGVR (SEQ ID NO: 17), GPQGIAGQR (SEQ ID NO: 18), PLGL (SEQ ID NO: 19), PLGLWA (SEQ ID NO: 20), GPLGVRG (SEQ ID NO: 21), PLGLAAV (SEQ ID NO: 22), GPILGR (SEQ ID NO: 23), PRVRLLG (SEQ ID NO: 24), APLANLG (SEQ ID NO: 25), PLAVNLG (SEQ ID NO: 26), ILGRLVREG (SEQ ID NO: 27), PLARSVLG (SEQ ID NO: 28), PILGIVGR (SEQ ID NO: 29), LRVSLGIVGR (SEQ ID NO: 30), PGVRVIG (SEQ ID NO: 31), PGILGISR (SEQ ID NO: 32), PLGILL (SEQ ID NO: 33), PQILGISR (SEQ ID NO: 34), PGILL (SEQ ID NO: 35), PLRLVS (SEQ ID NO: 36), PRVRLLG (SEQ ID NO: 37), CREIIGHR (SEQ ID NO: 38), RRRRR (SEQ ID NO: 39), RGL, KLEGGVR (SEQ ID NO: 40), WHHLR (SEQ ID NO: 41), RRRFMW (SEQ ID NO: 42), and/or RHRHRHRHRHRK (SEQ ID NO: 43). Alternatively, the cleavable entity may have at least 80% sequence identity, at least 85% sequence identity, at least 90% sequence identity, at least 95% sequence identity, at least 99% sequence identity, or be identical (e.g., 100% sequence identity) to one of SEQ ID NOs: 1-43. In another alternative, the polymer may be conjugated to a lipid, such as prostaglandin (including but not limited to PGD₂, PGE₂, and PGF₂).

The cleavable entity-polymer conjugate refers to a molecule wherein the previously described polymer is conjugated (e.g., covalently linked) to the previously described cleavable entity. In particular, the cleavable entity is linked to a terminal end of the polymer. The cleavable entity and polymer may be conjugated to each other through conventional means. For example, the cleavable entity may be attached to the polymer via a carbodiimide reaction, a maleimide-thiol reaction, click chemistry, and biotin-streptavidin to produce the peptide-polymer conjugate. The precise chemical reaction useful for preparing the cleavable-peptide polymer conjugate depends on several factors, including the type of materials used, whether the peptide or protein has additional modifications at the N- or C-terminals, and/or whether a linker is involved (e.g., a conjugating moiety). The polymers, peptides, or portions thereof may contain, or be modified to contain, appropriate functional groups (e.g., —OH, —COOH, —NH₂, —SH, azide, alkyne, and the like) for binding to the targeting moieties that have, or are modified to have, suitable functional groups.

The cleavable entity-polymer conjugate may protect the nanocomposite particle from degradation in the biological environment. However, when the cleavable entity is cleaved, such as from enzymatic activity by an MMP or an ADAMTS, the polymer is no longer protected from biodegradation, which leads to the breakdown of the nanocomposite particle and release of the magnetic nanoparticle and/or active agent into the local milieu (e.g., the organ, tissue, ECM, and/or tumor).

Alternatively, the cleavable entity refers to moieties which may not be cleaved, but instead undergo a conformational change upon exposure to a biological environment, such as arginase 1, iNOS, targets for 15-PGDH, reactive oxygen species (ROS), nitric oxide (NO), and/or an enzyme or other molecule that can induce the conformational change. The conformational change may then lead to biological degradation of the polymer and subsequent disintegration of the nanocomposite particle, thus releasing the magnetic nanoparticle and/or active agent into the local milieu.

Alternatively, the cleavable entity may be a molecule that responds to (e.g., undergoes a conformational change) reactive oxygen species (ROS). The ROS-responsive entity may be thiolether-containing polymers, thioketal-containing polymers, selenium-based polymers, tellurium-based polymers, proline oligomers (where the side chain of the oligomer is oxidized by ROS to produce carbonyl derivatives), polysaccharides, aminoacrylate, peroxalate esters and/or curcumin.

In another alternative, the polymer may be conjugated to a molecule that interacts with nitrogen oxide (NO). Such compounds include, but are not limited to, superoxide (O2-, hemoglobin, guanylyl cyclase, S-nitrosothiols (S-NO), (2-phenyl-4,4,5,5-tetramethylimidazoline-1-oxyl-3-oxide) (PTIO), hemoglobin derivatives (e.g., pyridoxalated hemoglobin polyoxyethylene conjugate (PHP)), low molecular weight iron compounds of diethylenetriaminepentaacetic acid, and diethyldithiocarbamate and ruthenium polyaminocarboxylate complexes.

As used herein, the “magnetic nanoparticle” refers to nanoparticles comprising compounds that include metal atoms, wherein the compounds possess magnetic properties. The magnetic nanoparticles are components of, and distinct from, the nanocomposite particles described in this application. The magnetic nanoparticles are highly monodisperse and able to respond to (e.g., be magnetized by) an external magnetic field. Moreover, the magnetic nanoparticles preferably can relax in changing magnetic fields, such as performed in MPI scanning, via Brownian relaxation. The magnetic nanoparticles useful in the nanocomposite particle are not particularly limited so long as they meet the above requirements. Exemplary magnetic nanoparticles include alloys and oxides of iron, cobalt, and nickel. Particular magnetic nanoparticles include iron oxides (e.g., Fe₃O₄, Fe₂O₃), iron-cobalt (FeCo) alloy, iron-nickel (FeNi) alloy, metal oxides (e.g., MnO, DyO), metal oxides comprising a divalent metal (e.g., MFe₂O₄, where M is a divalent metal, including, Ba²⁺, Cu²⁺, Co²⁺, Zn²⁺, Ni²⁺, Mn²⁺, and Mg²⁺), and pure iron (Fe). Possible divalent metal nanomaterials thus include BeFe₂O₄, CuFe₂O₄, CoFe₂O₄, ZnFe₂O₄, NiFe₂O₄, MnFe₂O₄, and MgFe₂O₄.

As used herein, the “active agent” refers to compounds which exert a clinically relevant effect when administered. The active agent may be an anti-cancer agent, an anti-inflammatory agent, an immunosuppressive agent, analgesic and/or anesthetic agents (e.g., pain medications), anti-allergy agents, and MMP inhibitors. The active agent may be a small molecule (e.g., chemical compound), protein (including peptides), nucleic acids (e.g., DNA, RNA, mRNA, siRNA, miRNA, lncRNA), cargo loaded on polymers (e.g., PLGA, PEI, chitosan, PLGA-PEG), and viral vectors. The active agent is loaded onto the nanocomposite particle in an amount sufficient to deliver a therapeutically effective amount of the active agent when the nanocomposite particle is administered to the subject.

The magnetic nanoparticle and active agent may generally be incorporated within the nanocomposite particle by mixing the magnetic nanoparticle and active agent with the cleavable entity-polymer conjugate in solution. The resulting nanocomposite particle is then isolated and washed prior to use.

### Nanocomposite Particles

The nanocomposite particles described herein comprise a polymer, a cleavable entity conjugated to the polymer, a magnetic nanoparticle, and a therapeutically effective amount of an active agent. The polymer of the nanocomposite particle may be one or more of PLGA, PEI, chitosan, PGA, PLA, poly(caprolactone), PEG, polyhydroxybutyrate, hyaluronic acid, polymethylmethacrylate, polyethylmethacrylate, polypropylene, collagen, gelatin, albumin, alginate, poly(methacrylic acid), poly(acid acrylic), poly(anhydrides), PEG-PLA, and PLGA-PEG-COOH. In particular, the polymer may be PLGA, such as acid-terminated PLGA having a lactide:glycolide ratio of about 65:35 and a molecular weight of about 24-38 kDa, acid-terminated PLGA having a lactide:glycolide ratio of about 75:25 and a molecular weight of about 75-115 kDa, PLGA having a lactide:glycolide ratio of about 50:50 and a molecular weight of about 4.2 kDa, PLGA having a lactide:glycolide ratio of about 50:50 and a molecular weight of about 24-38 kDa, PLGA having a lactide:glycolide ratio of about 50:50 and a molecular weight of about 38-54 kDa, poly(D,L-lactide-co-glycolide)-b-poly(ethylene glycol)-carboxylic acid having a lactide:glycolide ratio of about 85:15 and a molecular weight of about 5-13 kDa, or polyglycolic acid/80:10 or polylactic acid:polyglycolic acid/90:10.

In some embodiments, the cleavable entity is an amino acid sequence that may be altered by an enzyme, such as cleavage by MMP and/or ADAMTS, or a conformation change with arginase 1, iNOS, targets for 15-PGDH, reactive oxygen species, and/or enzymes that change the conformation of the cleavable entity. Alternatively, the cleavable entity may be an amino acid sequence that undergoes a conformational (three-dimensional) change following contact with an enzyme, such as arginase I or other enzymes that induce a conformational change, or a compound, such as iNOS, reactive oxygen species (ROS), and/or targets for 15-PGDH. The cleavable entity may be cleaved by a particular MMP, such as MMP-1, MMP-2, MMP-7, MMP-9, MMP-13, MMP-14, MMP-15, and/or MTI-MMP. The sequence of the cleavable entity my have at least 75% sequence identity to one or more of GPLGIAGQ (SEQ ID NO: 1), PVGLIG (SEQ ID NO: 2), PLGLAG (SEQ ID NO: 3), GGGVPLSLYSGGGG (SEQ ID NO: 4), VLVPMAMMAS (SEQ ID NO: 5), GPLGMRGLGK (SEQ ID NO: 6), VPLSLYSGCG (SEQ ID NO: 7), GQPGIFGQ (SEQ ID NO: 8), RPLALWRS (SEQ ID NO: 9), GPVGLIGK (SEQ ID NO: 10), HPVGLLAR (SEQ ID NO: 11), DGPLGVC (SEQ ID NO: 12), GPQGIAG (SEQ ID NO: 13), GRIGFLR (SEQ ID NO: 14), RIGFLR (SEQ ID NO: 15), GPLGV (SEQ ID NO: 16), PLGVR (SEQ ID NO: 17), GPQGIAGQR (SEQ ID NO: 18), PLGL (SEQ ID NO: 19), PLGLWA (SEQ ID NO: 20), GPLGVRG (SEQ ID NO: 21) PLGLAAV (SEQ ID NO: 22), GPILGR (SEQ ID NO: 23), PRVRLLG (SEQ ID NO: 24), APLANLG (SEQ ID NO: 25), PLAVNLG (SEQ ID NO: 26), ILGRLVREG (SEQ ID NO: 27), PLARSVLG (SEQ ID NO: 28), PILGIVGR (SEQ ID NO: 29), LRVSLGIVGR (SEQ ID NO: 30), PGVRVIG (SEQ ID NO: 31), PGILGISR (SEQ ID NO: 32), PLGILL (SEQ ID NO: 33), PQILGISR (SEQ ID NO: 34), PGILL (SEQ ID NO: 35), PLRLVS (SEQ ID NO: 36), PRVRLLG (SEQ ID NO: 37), CREIIGHR (SEQ ID NO: 38), RRRRR (SEQ ID NO: 39), RGL, KLEGGVR (SEQ ID NO: 40), WHHLR (SEQ ID NO: 41), RRRFMW (SEQ ID NO: 42), and/or RHRHRHRHRHRK (SEQ ID NO: 43). Alternatively, the sequence of the cleavable entity conjugated to the polymer may have at least 80%, at least 85%, at least 90%, at least 95%, or at least 99% sequence identity to one or more of SEQ ID NOs: 1-43. In a particular embodiment the cleavable entity conjugated to the polymer may be one of SEQ ID NOs: 1-43. In a particular embodiment, the cleavable entity may be GPLGIAGQ (SEQ ID NO: 1). The target for 15-PGDH may be PGE₂.

In some embodiments, the magnetic nanoparticle of the nanocomposite particle may have low size variation (e.g., of <5%) for use in MPI. For example, the magnetic nanoparticle may be Fe₃O₄, Fe₂O₃, FeCo, FeNi, MnO, and DyO. Alternatively, the magnetic nanoparticle may be of the formula Mfe₂O₄, where M is a divalent metal. In a particular embodiment, the magnetic nanoparticle is Fe₃O₄.

In some embodiments, the active agent incorporated within the nanocomposite particle may be an anti-cancer agent, an anti-inflammatory agent, an immunosuppressive agent, and MMP inhibitors. The active agent may be a small molecule (e.g., chemical compound), protein (including peptides), nucleic acid (e.g., DNA or RNA), or viral vector. When the active agent is an anti-cancer agent, the active agent may be one or more of, actinomycin, alitretinoin, all-trans retinoic acid, altretamine, azacitidine, azathioprine, belotecan, bendamustine, bexarotene, bleomycin, bortezomib, busulfan, cabazitaxel, camptothecin, carboplatin, carboquone, carmustine, capecitabine, cisplatin, chlorambucil, chlormethine, chlorozotocin, cyclophosphamide, cytarabine, dacarbazine, daunorubicin, docetaxel, doxifluridine, doxorubicin, epirubicin, epothilone, erlotinib, etoposide, exatecan, fluorouracil, fotemustine, gefitinib, gemcitabine, gimatecan, hydroxyurea, idarubicin, ifosfamide doxorubicin, imatinib, irinotecan, ixabepilone, larotaxel, lomustine, melphalan, melphalan flufenamide, mercaptopurine, methotrexate, mitobronitol, mitomycin C, mitoxantrone, nimustine, nitrosoureas, oxaliplatin, paclitaxel, pemetrexed, pipobroman, ranimustine, romidepsin, semustine, streptozotocin, tafluposide, taxotere, temozolomide, tesetaxel, teniposide, thiotepa, tioguanine, topotecan, treosulfan, tretinoin, triaziquone, triethylenemelamine, valrubicin, vemurafenib, vinblastine, vincristine, vindesine, vinorelbine, vismodegib, and vorinostat. In a specific embodiment, the active agent is doxorubicin. When the active agent is an MMP inhibitor, the active agent may be hydroxamic acid, marimastat, batimastat, thiols (such as rebimastat, tanomastat, captopril, Fosfomycin, and PD166793), carbamoylphosphonate, hydrazines (such as SB-3CT), bis(dioxopiperazine, beta lactams, nitrogenous substances (such as Ro 28-653), tetracycline-based inhibitors (such as doxycycline hyclate, COL-3, or metastat), hydroxamate-based inhibitors (such as CGS 27023A and prinomastat), and tissue inhibitors of matrix metalloproteases (TIMPs), including TIMP-1, TIMP-2, TIMP-3, and/or TIMP-4. In another specific embodiment, the active agent is doxycycline hyclate or prinomastat.

In a preferred embodiment, the nanocomposite particle comprises PLGA, GPLGIAGQ (SEQ ID NO: 1) conjugated to the PLGA, Fe₃O₄, and doxorubicin. In another preferred embodiment, the nanocomposite particle comprises PLGA, GPLGIAGQ (SEQ ID NO: 1) conjugated to the PLGA, Fe₃O₄, and doxycycline hyclate. In yet another preferred embodiment, the nanocomposite particle comprises PLGA, GPLGIAGQ (SEQ ID NO: 1) conjugated to the PLGA, Fe₃O₄, and prinomastat.

### Pharmaceutical Compositions

Further described herein are pharmaceutical compositions comprising the nanocomposite particle described above and one or more pharmaceutically acceptable excipients. One of skill in the art would appreciate that the choice of any one excipient may influence the choice of any other excipient. For example, the choice of a particular excipient may preclude the use of one or more additional excipients because the combination of excipients would produce undesirable effects. One of skill in the art would be able to determine empirically which excipients, if any, to include in the formulations or compositions disclosed herein. Excipients may include, but are not limited to, co-solvents, solubilizing agents, buffers, pH adjusting agents, bulking agents, surfactants, encapsulating agents, tonicity-adjusting agents, stabilizing agents, protectants, viscosity modifiers, and combinations thereof.

The pharmaceutical composition comprising the nanocomposite particle may be formulated for their intended route of administration. The composition may be formulated for systemic, peripheral, proximal to the location of a target site (e.g., near a tumor), and/or intratumorally. Suitable routes of administration will be apparent to those of skill in the art, depending on the type of condition to be prevented or treated, the therapeutically-active agent used, and/or the target cell population or tissue. Various acceptable methods of administration include, but are not limited to, intravenous administration, intraperitoneal administration, intramuscular administration, intranodal administration, intracoronary administration, intraarterial administration (e.g., into a carotid artery), subcutaneous administration, retroorbital administration, transdermal delivery, intratracheal administration, subcutaneous administration, intraarticular administration, intraventricular administration, inhalation (e.g., aerosol), intracranial, intraspinal, intraocular, aural, intranasal, oral, pulmonary administration, impregnation of a catheter, and direct injection into a tissue. In one aspect, routes of administration include: intravenous, intraperitoneal, subcutaneous, intradermal, intranodal, intramuscular, transdermal, inhaled, intranasal, oral, intraocular, intraarticular, intracranial, and intraspinal. Parenteral delivery can include intradermal, intramuscular, intraperitoneal, intrapleural, intrapulmonary, intravenous, subcutaneous, atrial catheter and venal catheter routes. Aural delivery can include ear drops, intranasal delivery can include nose drops or intranasal injection, and intraocular delivery can include eye drops. Aerosol (inhalation) delivery can also be performed using methods standard in the art. Other routes of administration that modulate mucosal immunity are useful in the treatment of viral infections. Such routes include bronchial, intradermal, intramuscular, intranasal, other inhalatory, rectal, subcutaneous, topical, transdermal, vaginal and urethral routes. In a particular embodiment the pharmaceutical composition is administered by injection, such as intratumorally.

### Methods of Treatment

Also described herein is a method for treating cancer in a subject comprising administering to the subject the nanocomposite particle described above. To treat cancer, the nanocomposite particle described above may be administered in a therapeutically effective amount. The subject may be symptomatic or asymptomatic. In an embodiment, the cancer to be treated is not particularly limited, and may be cancer of the breast, brain, pancreas, prostate, ovary, lung, brain, bladder, testicle, lymph and lymph nodes, muscle, liver, skin, mouth, intestine, colon, and stomach. The nanocomposite polymer may also be for treating inflammatory diseases (e.g., arthritis, neurodegeneration, atherosclerosis, etc.), treating heart diseases, treating immunosuppressive diseases, enhancing wound healing, and/or inducing or decreasing tissue/bone remodeling.

Where the nanocomposite particle is to be administered, any suitable route of administration may be used, including but not limited to intravenous injection, intramuscular injection, subcutaneous injection, and intratumoral injection. Therapeutically effective amounts of these nanocomposite particles relate to the amount of active agent that can be loaded onto the nanocomposite particle and released upon biodegradation, as well as the identity of the polymer used and its degradation kinetics. The amount of active agent loaded onto the nanocomposite polymer depends upon the identity of the active agent. When a nucleic acid (e.g., DNA, RNA, mRNA, lncRNA, etc.) is the active agent, the amount to be loaded onto the nanocomposite material may include (but is not limited to) 10 ng, 50 ng, 100 ng, 500 ng, 1 μg, 5 μg, 10 μg, 50 μg, 100 μg, 500 μg, 1 mg, 5 mg, 10 mg, 50 mg, 100 mg, or more. The amount of therapeutically effective viral vector loaded onto the nanocomposite particle may be, but is not limited to, 1×10⁷ viral particles (VPs), 5×10⁷ VPs, 1×10⁸ VPs, 5×10⁸ VPs, 1×10⁹ VPs, 5×10⁹ VPs, 1×10¹⁰ VPs, or more than 1×10¹⁰ VPs. In some embodiments, the amount of nanocomposite particle to be administered may be between about 0.001 mg/kg and 100 mg/kg, between about 0.01 mg/kg and about 10 mg/kg, or between about 0.1 mg/kg and about 1.0 mg/kg. In a particular embodiment, the nanocomposite particle may be administered at a dose of about 0.12 mg/kg.

### Examples

The following example is provided to further illustrate the fusion peptide disclosed herein but should not be construed as in any way limiting its scope.

MANUFACTURE OF PLGA-PEPTIDE CONJUGATE: PLGA-peptide conjugate was prepared using a previously described method by Pan et al. (Chem Commun, 2018; 54 (79)). Briefly, N,N′-dicyclohexylcarbodiimide (DCC) (1 mmol) and N-hydroxysuccinimide (NHS) (1 mmol) were mixed. PLGA-COOH (1 mmol) in chloroform was added to the DCC/NHS mixture under an inert atmosphere. The reaction mixture was stirred for 24 hr to activate the carboxyl groups of PLGA. Then, the activated carboxyl of PLGA-COOH and the MMP-2-cleavable entity GPLGIAGQ (SEQ ID NO: 1) (1.2 mmol) were mixed in carbonate buffer at 4° C. overnight (pH 8.5) under stirring to generate the active peptide conjugate. The resultant solution was dialyzed (MW 3.5 kDa) against distilled water and freeze-dried to obtain a white solid. Similarly, PLGA-inactive peptide conjugate was prepared using the above-mentioned method. The inactive peptide is not cleavable by MMP-2 and has the sequence GPLAGAGQ (SEQ ID NO: 22).

MANUFACTURE OF PLGA-PEPTIDE NANOCOMPOSITE PARTICLE: Nanocomposite particles were prepared with the blend of PLGA-peptide (either cleavable or uncleavable entity) and PLGA at the weight ratio of 5:1 through a nanoprecipitation method. PLGA-peptide (25 mg), PLGA (5 mg), DOX (1 mg) and iron oxide (Fe₃O₄) nanoparticles (70 μL) were dissolved in DMSO (4 mL) under stirring. Next, the mixture was added dropwise to 0.1% of polyvinyl alcohol (30 mL) using 1 mL pipette under continuous stirring. Then, the reaction solution was stirred overnight. The solution was then washed using 10 kDa centrifuge filters and the nanoparticles were washed twice with PBS (pH 7.4) to remove free DOX. The nanoparticles were centrifuged again to collect the DNCa. Further, the washed DNCa were freeze-dried using the Freezone system (Labconco) for 24 hours and stored until further use. DOX and iron oxide-loaded PLGA-inactive peptide nanocomposite particles (DNCi) were prepared using similar method. Nanocomposite particles without doxorubicin (FNCa) were also prepared using the similar method.

CHARACTERIZATION OF PLGA-PEPTIDE CONJUGATE AND NANOCOMPOSITE PARTICLES: PLGA-peptide conjugate was dissolved in water or DMSO, and UV-Vis spectra was taken using a UV-Vis spectrometer (Shimadzu). The size and zeta potential of DNCa, DNCi, and FNCa were evaluated by a zeta potential and particle size analyzer (Malvern Zetasizer NanoZS DLS instrument). The morphology of nanocomposite particle was verified by scanning electron microscope (SEM). Briefly, nanocomposite nanoparticles were mounted on aluminum stubs using high vacuum carbon tabs (SPI Supplies, West Chester, PA). Later, samples were coated with iridium (approximate thickness ~2.7 nm) in a Quorum Technologies/Electron Microscopy Sciences Q150T turbo pumped sputter coater (Quorum Technologies, Laughton, East Sussex, England BN8 6BN) purged with argon gas. Samples were examined in a JEOL 7500F (Field emission) scanning electron microscope (JEOL Ltd., Tokyo, Japan). Finally, samples were imaged at an accelerating voltage of 5 kV.

Drug loading was evaluated by weighing the nanocomposite particles (1 mg) and dissolved in DMSO and sonicated for half an hour. The concentration of DOX in the nanocomposite particle was determined from the corresponding blank-subtracted by fluorescence measurement (excitation and emission wavelengths of 485 nm and 590 nm, respectively) using a plate reader with UV-Vis spectra (Shimadzu UV-2600 spectrometer). Drug loading content was quantified by referring to a standard curve of DOX. Drug loading content (DLC) was calculated using following formula (where “NPS” refers to the nanocomposite particle):

$$DL \% = \frac{weight of Dox in NPS}{weight of NPS} * 100 .$$

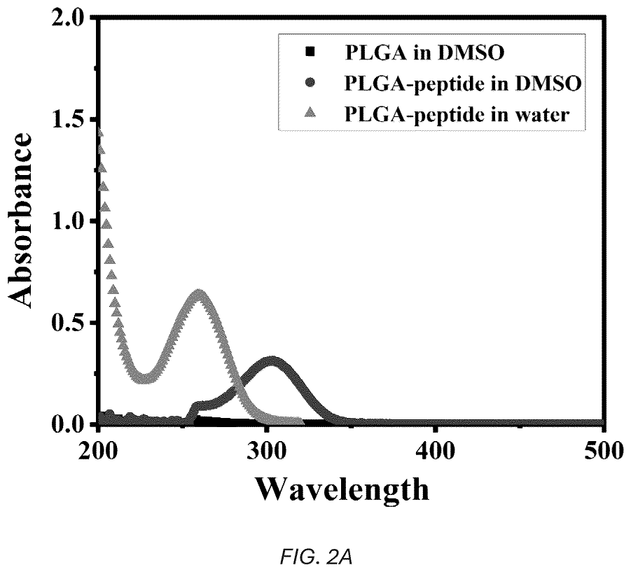

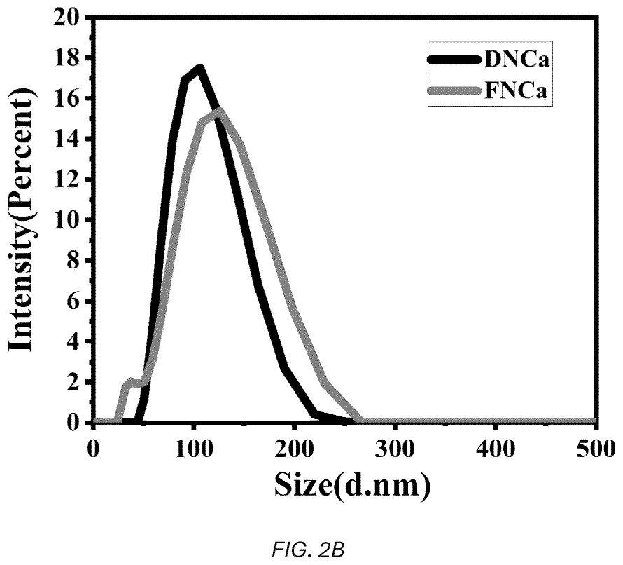

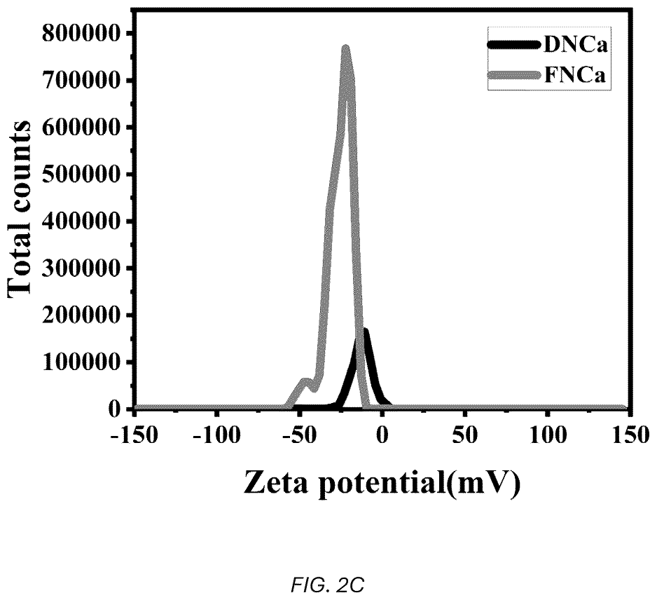

FIG. 2A shows the analysis of the PLGA-peptide peaks as characterized by UV-Vis spectroscopy in water and DMSO. The size of DNCa was uniform. The nanocomposite particles had average diameters of 96.9 and 94.4 nm with poly dispersity index of 0.076 and 0.14 for DNCa and FNCa, respectively, as measured by dynamic light scattering (DLS) measurement (FIG. 2B). The zeta potential was −10.87 and −22.34 mV, respectively (FIG. 2C).

DRUG RELEASE AND MAGNETIC PARTICLE IMAGING OF DNCa: The release of DOX from the DNCa was measured in PBS (pH 6.5) at 37° C. in the presence or absence of MMP-2. Samples were dispersed in PBS (10 mL, 0.1 M, pH 6.5) with or without MMP-2 (at 0, 0.05, 0.25, 0.5, 1.0 mg/mL), and were immersed into a dialysis bag (SPECTRA/POR3; MW 10 kDa). Afterwards, the bag was immersed into PBS (500 mL, 0.1 M, pH 6.5) at 37° C. while stirring at 200 rpm. At predetermined time intervals (0, 1, 3, 5, 8, 24, 48, and 72 hr), the release medium (1 mL) was removed and the amount of DOX in the solution was determined by fluorescence measurement (excitation and emission wavelengths of 485 nm and 590 nm, respectively) using a plate reader with fluorescence spectra. The concentration of the released DOX from DNCa was calculated using a serial dilution of a DOX standard solution.

DNCa serves dual purpose as a drug delivery system and an MPI-based drug release monitoring agent. Drug release and MPI release from DNCa was evaluated by dispersing DNCa (1 mg) in 200 μL of phosphate buffer (pH 6.5) with or without MMP-2 (0.5 mg/ml). The solutions in tubes were imaged at different time points (0, 1, 3, 5, 8, 24, and 48 hr) by MPI. The concentration of the released DOX in the solution was determined by fluorescence measurement described above.

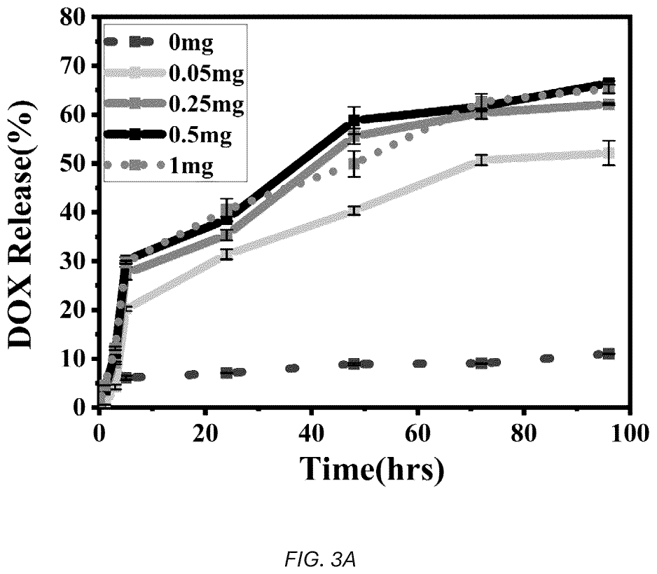

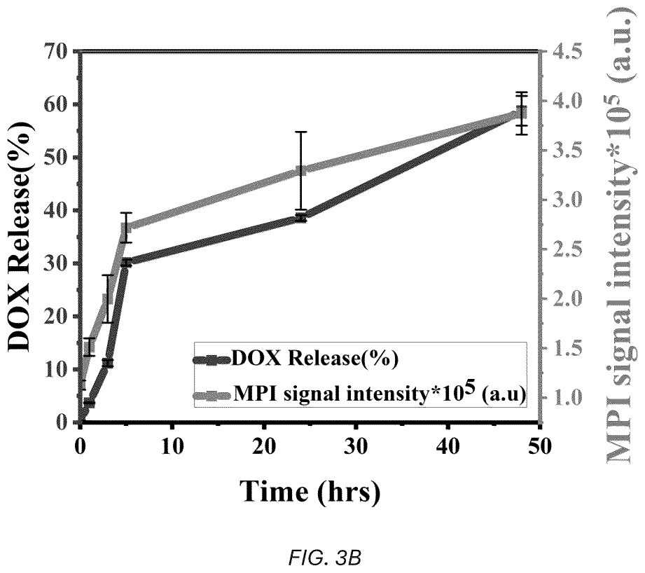

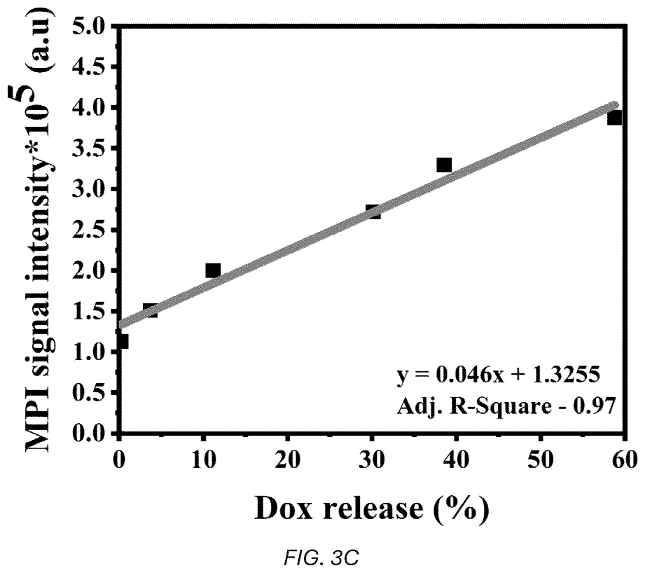

To investigate drug release monitoring from DNCa, different collagenase (which includes MMP2) enzyme concentrations (0, 0.05, 0.25, 0.5, and 1 mg/ml) were dispersed in phosphate buffer solution (pH 6.5) to simulate the intracellular environment pH. DNCa dispersion was kept at 37° C. for 96 h. The release of DOX at different time points was measured by fluorescence (emission 470 nm; excitation; 595 nm) FIG. 3A shows the in vitro drug release profiles of nanoparticles and a continuous release pattern with an initial burst drug release. In the absence of collagenase enzyme (0 mg/ml), drug release from the DNCa was slow in the first 24 hr, and the percentage release of DOX from DNCa was around 11% at 96 hr. In contrast, incubation with collagenase at higher concentrations showed increased enzyme dependent MPI signal. FIG. 3A showed that increasing enzyme concentration from 0 mg/ml to 1 mg/mL leads to faster drug release. At 0.5 mg/ml collagenase concentration showed approximately 66.45% of the DOX and appears to be saturation concentration for drug release. These results demonstrate the MMP-2 responsive drug release of the DNCa.

FIG. 3B shows a correlation between the DNCa MPI signal change and the DOX release rate. The MPI signal change correlated with the release of a drug from DNCa. The DOX release from the DNCa started with a rapid increase until 8 hr, followed by a more gradual release over time (from 8 to 48 hr). A linear correlation with R²=0.97 was observed when the percent DOX release and MPI signals were analyzed across multiple time points (FIG. 3C). The linearity in the relationship between DOX release and MPI signal facilitates the quantification monitoring of drug release based on local MMP-2 levels.

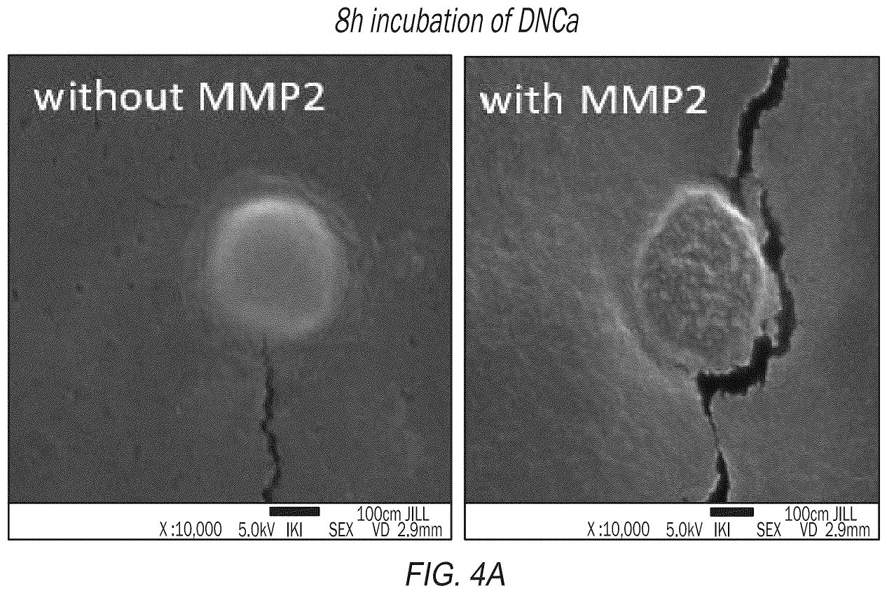

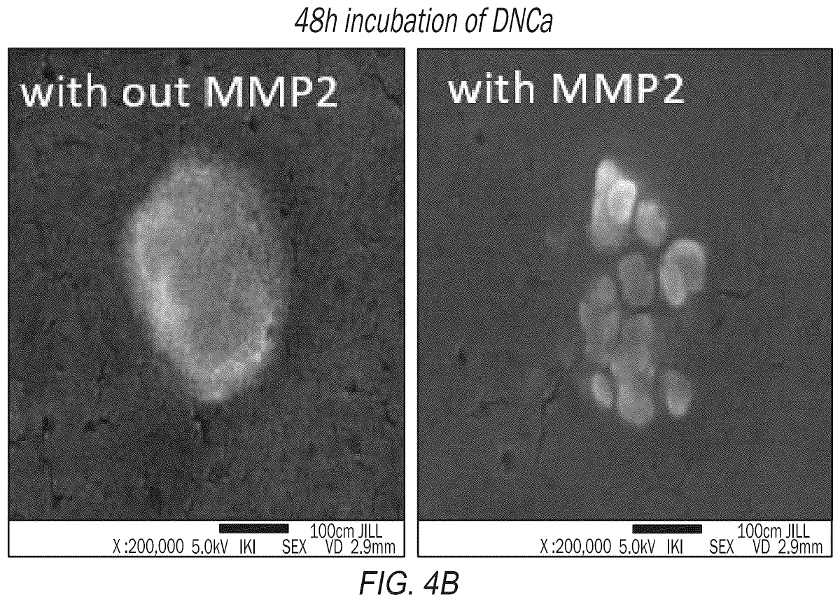

The DNCa was imaged with SEM to examine the cause of the MPI signal change and drug release. (FIGS. 4A and 4B). DNCa were degraded while they were incubated with collagenase (including MMP-2) (0.5 mg/ml) in buffer (pH 6.5) for 8 hr. (FIG. 4A) The surface of DNCa became more irregular and rougher as compared to DNCa that were incubated without collagenase (including MMP-2), which likely caused the MPI signal to change. At longer incubation time of 48 hr, the nanoparticles appeared degraded and disassembled. (FIG. 4B)

TUMOR XENOGRAFTS: All animal experiments were performed in compliance with the guidelines for the care and use of laboratory animals of Michigan State University requirements and according to the IACUC protocol approved by the Animal Care. 4T1 breast cancer cells (ATCC) were prepared by incubation with 0.05% trypsin-EDTA and then collected by centrifugation and resuspended in sterile PBS. 4T1 cells (5×10⁶ cells per mouse) were implanted into the mammary fat pads of 6-8 weeks old female mice. When the tumors reached a volume of 100-120 mm³ (7 days after tumor cell implantation), the mice were randomly divided into 2 groups (n=4): DNCa treated group and DNCi treated group (control group). 4T1 tumor-bearing mice were injected intratumorally with nanoparticles at a concentration of 5 μg/50 μl PBS. The mice were imaged with MPI at time points of 0, 1, 3, 5, 8, 24, 48, 72 and 96 hours. Tumor volume was measured using the equation:

$$Tumor volume ( {mm}^{3} ) \% = \frac{length \times {width}^{2}}{2}$$

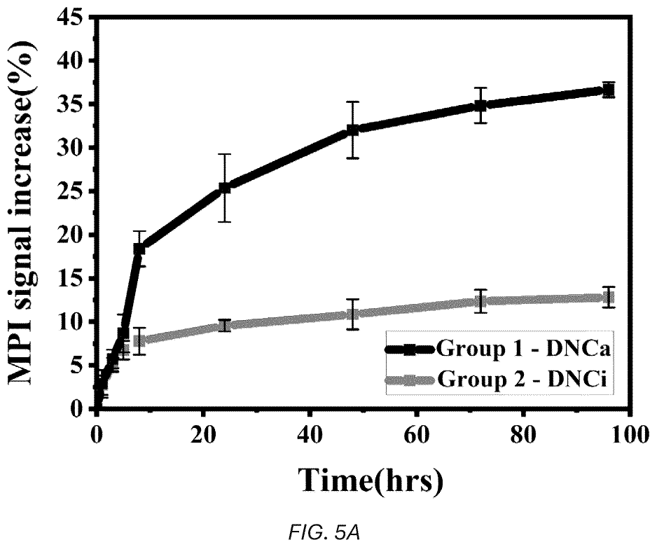

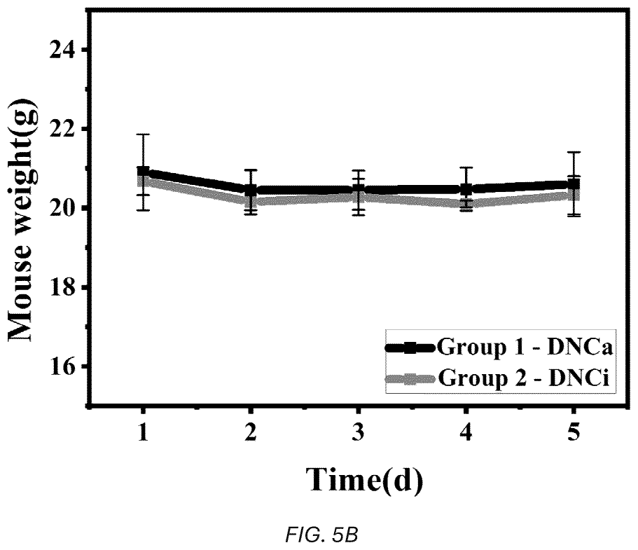

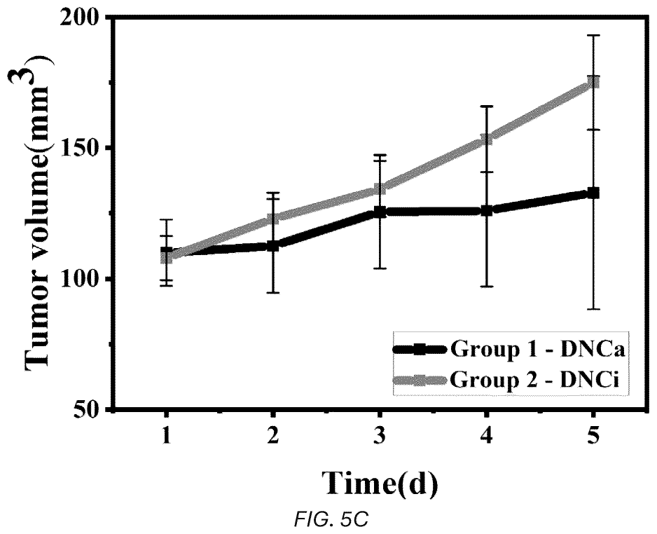

After successfully monitoring the MPI signal and evaluating the linear relationship between MPI signal and DOX in buffer solution (pH 6.5), change in MPI signal was monitored in animal models. A single dose of nanocomposite particles was intratumorally injected into the implanted orthotropic breast cancer 4T1 murine model (n=4 per group, DNCa and DNCi control). The imaging was performed at different time points (0 to 96 h) to observe changes in signal intensity in tumor region. The MPI signal intensity increased gradually over 96 hours (FIG. 5A), which showed that the drug was slowly released from the DNCa. The release from DNCa showed biphasic behavior, with an initial, rapid release of DOX in the first few hours followed by a slower and steadier release over the next days. The release profile was consistent with the MPI signal change in buffer as shown earlier in FIG. 3B. In contrast the DNCi showed very little change (12.80%) in signal over time (0 to 96 hr) as compared to DNCa which showed higher signal increase of 36.65%. The average tumor size of the DNCa group increased more slowly than the average tumor size in the DNCi group (FIG. 5C). On day 0 of imaging, the tumor volume was 109 and 107 mm³ for DNCa and DNCi treated group which increased to 132 and 174 mm³ respectively on day 5 (96 hr). There was no significant change in body weight as observed in both groups (FIG. 5B).

The use of the terms “a” and “an” and “the” and “at least one” and similar referents in the context of describing the nanocomposite polymer and related uses (especially in the context of the following claims) are to be construed to cover both the singular and the plural, unless otherwise indicated herein or clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Particular embodiments of the fusion peptide are described herein, including the best mode known to the inventors for carrying out the invention. Variations of those particular embodiments may become apparent to those of ordinary skill in the art upon reading the foregoing description. The inventors expect skilled artisans to employ such variations as appropriate, and the inventors intend for the fusion peptide to be practiced otherwise than as specifically described herein. Accordingly, the fusion peptide described herein includes all modifications and equivalents of the subject matter recited in the claims appended hereto as permitted by applicable law. Moreover, any combination of the above-described elements in all possible variations thereof is encompassed by the described fusion peptide unless otherwise indicated herein or otherwise clearly contradicted by context.

1. A nanocomposite particle comprising
a polymer;
a cleavable entity conjugated to the polymer;
a magnetic nanoparticle; and
a therapeutically effective amount of an active agent.

2. The nanocomposite particle according to claim 1, wherein the polymer is selected from the group consisting of poly(lactic-co-glycolic acid) (PLGA), polyethylenimine (PEI), chitosan, polyglycolic acid (PGA), polylactic acid (PLA), poly(caprolactone), polyethylene glycol (PEG), polyhydroxybutyrate, hyaluronic acid, polymethylmethacrylate, polyethylmethacrylate, polypropylene, collagen, gelatin, albumin, alginate, poly(methacrylic acid), poly(acid acrylic), poly(anhydrides), PEG-PLA, and PLGA-PEG-COOH.

3. The nanocomposite particle according to claim 1, wherein the polymer is PLGA.

4. The nanocomposite particle according to claim 1, wherein the polymer is selected from the group consisting of acid-terminated PLGA having a lactide:glycolide ratio of about 65:35 and a molecular weight of about 24-38 kDa, acid-terminated PLGA having a lactide:glycolide ratio of about 75:25 and a molecular weight of about 75-115 kDa, PLGA having a lactide:glycolide ratio of about 50:50 and a molecular weight of about 4.2 kDa, PLGA having a lactide:glycolide ratio of about 50:50 and a molecular weight of about 24-38 kDa, PLGA having a lactide:glycolide ratio of about 50:50 and a molecular weight of about 38-54 kDa, poly(D,L-lactide-co-glycolide)-b-poly(ethylene glycol)-carboxylic acid having a lactide:glycolide ratio of about 85:15 and a molecular weight of about 5-13 kDa, and polyglycolic acid/80:10 or polylactic acid:polyglycolic acid/90:10.

5. The nanocomposite particle according to claim 1, wherein the cleavable entity comprises an amino acid sequence that is cleaved by an enzyme selected from the group consisting of matrix metalloprotease (MMP) and a disintegrin and metalloproteinase with a thrombospondin motif (ADAMTS), or undergoes a conformational change by an enzyme or molecule selected from the group consisting of arginase I, iNOS, reactive oxygen species, nitric oxide, and targets for 15-PGDH.

6. The nanocomposite particle according to claim 5, wherein the MMP is selected from the group consisting of MMP-1, MMP-2, MMP-7, MMP-9, MMP-13, MMP-14, MMP-15, and MTI-MMP.

7. The nanocomposite particle according to claim 6, wherein the cleavable entity comprises an amino acid sequence selected from the group consisting of GPLGIAGQ (SEQ ID NO: 1), PVGLIG (SEQ ID NO: 2), PLGLAG (SEQ ID NO: 3), GGGVPLSLYSGGGG (SEQ ID NO: 4), VLVPMAMMAS (SEQ ID NO: 5), GPLGMRGLGK (SEQ ID NO: 6), VPLSLYSGCG (SEQ ID NO: 7), GQPGIFGQ (SEQ ID NO: 8), RPLALWRS (SEQ ID NO: 9), GPVGLIGK (SEQ ID NO: 10), HPVGLLAR (SEQ ID NO: 11), DGPLGVC (SEQ ID NO: 12), GPQGIAG (SEQ ID NO: 13), GRIGFLR (SEQ ID NO: 14), RIGFLR (SEQ ID NO: 15), GPLGV (SEQ ID NO: 16), PLGVR (SEQ ID NO: 17), GPQGIAGQR (SEQ ID NO: 18), PLGL (SEQ ID NO: 19), PLGLWA (SEQ ID NO: 20), and GPLGVRG (SEQ ID NO: 21), PLGLAAV (SEQ ID NO: 22), GPILGR (SEQ ID NO: 23), PRVRLLG (SEQ ID NO: 24), APLANLG (SEQ ID NO: 25), PLAVNLG (SEQ ID NO: 26), ILGRLVREG (SEQ ID NO: 27), PLARSVLG (SEQ ID NO: 28), PILGIVGR (SEQ ID NO: 29), LRVSLGIVGR (SEQ ID NO: 30), PGVRVIG (SEQ ID NO: 31), PGILGISR (SEQ ID NO: 32), PLGILL (SEQ ID NO: 33), PQILGISR (SEQ ID NO: 34), PGILL (SEQ ID NO: 35), PLRLVS (SEQ ID NO: 36), PRVRLLG (SEQ ID NO: 37), CREIIGHR (SEQ ID NO: 38), RRRRR (SEQ ID NO: 39), RGL, KLEGGVR (SEQ ID NO: 40), WHHLR (SEQ ID NO: 41), RRRFMW (SEQ ID NO: 42), and RHRHRHRHRHRK (SEQ ID NO: 43).

8. The nanocomposite particle according to claim 6, wherein the cleavable entity is GPLGIAGQ (SEQ ID NO: 1).

9. The nanocomposite particle according to claim 1, wherein the magnetic nanoparticle is selected from the group consisting of Fe₃O₄, Fe₂O₃, FeCo, FeNi, MnO, and DyO.

10. The nanocomposite particle according to claim 9, wherein the magnetic nanoparticle is Fe₃O₄.

11. The nanocomposite particle according to claim 1, wherein the active agent is selected from the group consisting of an anti-cancer agent, an anti-inflammatory agent, an immunosuppressive agent, and an MMP inhibitor.

12. The nanocomposite particle according to claim 11, wherein the active agent is an anti-cancer agent selected from the group consisting of, actinomycin, alitretinoin, all-trans retinoic acid, altretamine, azacitidine, azathioprine, belotecan, bendamustine, bexarotene, bleomycin, bortezomib, busulfan, cabazitaxel, camptothecin, carboplatin, carboquone, carmustine, capecitabine, cisplatin, chlorambucil, chlormethine, chlorozotocin, cyclophosphamide, cytarabine, dacarbazine, daunorubicin, docetaxel, doxifluridine, doxorubicin, epirubicin, epothilone, erlotinib, etoposide, exatecan, fluorouracil, fotemustine, gefitinib, gemcitabine, gimatecan, hydroxyurea, idarubicin, ifosfamide doxorubicin, imatinib, irinotecan, ixabepilone, larotaxel, lomustine, melphalan, melphalan flufenamide, mercaptopurine, methotrexate, mitobronitol, mitomycin C, mitoxantrone, nimustine, nitrosoureas, oxaliplatin, paclitaxel, pemetrexed, pipobroman, ranimustine, romidepsin, semustine, streptozotocin, tafluposide, taxotere, temozolomide, tesetaxel, teniposide, thiotepa, tioguanine, topotecan, treosulfan, tretinoin, triaziquone, triethylenemelamine, valrubicin, vemurafenib, vinblastine, vincristine, vindesine, vinorelbine, vismodegib, and vorinostat.

13. The nanocomposite particle according to claim 12, wherein the active agent is doxorubicin.

14. The nanocomposite particle according to claim 1, wherein
the polymer is PLGA;
the cleavable entity is GPLGIAGQ (SEQ ID NO: 1);
the magnetic nanoparticle is Fe₃O₄; and
the active agent is doxorubicin.

15. The nanocomposite particle according to claim 1, wherein
the polymer is PLGA;
the cleavable entity is GPLGIAGQ (SEQ ID NO: 1);
the magnetic nanoparticle is Fe₃O₄; and
the active agent is doxycycline hyclate.

16. The nanocomposite particle according to claim 1, wherein
the polymer is PLGA;
the cleavable entity is GPLGIAGQ (SEQ ID NO: 1);
the magnetic nanoparticle is Fe₃O₄; and
the active agent is prinomastat.

17. A pharmaceutical composition comprising the nanocomposite particle according to claim 1 and one or more pharmaceutically acceptable excipients.

18. The pharmaceutical composition according to claim 17, wherein the pharmaceutical composition is formulated for injection.

19. The pharmaceutical composition according to claim 17, wherein the one or more pharmaceutically acceptable excipients is selected from the group consisting of a co-solvent, a solubilizing agents, a buffer, a pH adjusting agent, a bulking agent, a surfactant, an encapsulating agent, a tonicity-adjusting agent, a stabilizing agent, a protectant, a viscosity modifier, and a combinations thereof.

20. A method for treating cancer in a subject in need thereof, the method comprising administering to the subject the pharmaceutical composition of claim 17.

21. The method according to claim 20, wherein the cancer is selected from the group consisting of cancer of the breast, brain, pancreas, prostate, ovary, lung, bladder, testicle, lymph and lymph nodes, muscle, liver, skin, mouth, intestine, colon, and stomach.

22. The method according to claim 21, wherein the nanocomposite particle is administered at a dose of about 0.12 mg/kg.
